(19) United States
(12) Patent Application Publication
(10) Pub. No.: US 2014/0370061 A9
(43) Pub. Date: Dec. 18, 2014

(54) COMBINATION OF ACTIVE INGREDIENTS FOR COSMETIC PREPARATIONS

(71) Applicant: Marcus Asam, Unterföhring (DE)

(72) Inventor: Marcus Asam, Unterföhring (DE)

(21) Appl. No.: 13/683,525

(22) Filed: Nov. 21, 2012

Related U.S. Application Data
(62) Division of application No. 12/214,154, filed on Jun. 16, 2008, now abandoned.

(30) Foreign Application Priority Data
Jun. 15, 2007 (DE) 10 2007 028 360.3

(51) Int. Cl. A61K 8/73; A61Q 19/00; A61Q 19/08

(52) U.S. Cl. CPC A61K 8/735; A61Q 19/08; A61Q 19/00
USPC 424/401; 514/54

(57) ABSTRACT

A combination of active substances for cosmetic preparations contains hyaluronic acid and at least one of its derivatives. At least 10 percent by weight is used in long-chain form with a molecular weight of at least 1,000,000 daltons. At least 2 percent by weight is used in short-chain form with a molecular weight not more than 200,000 daltons.

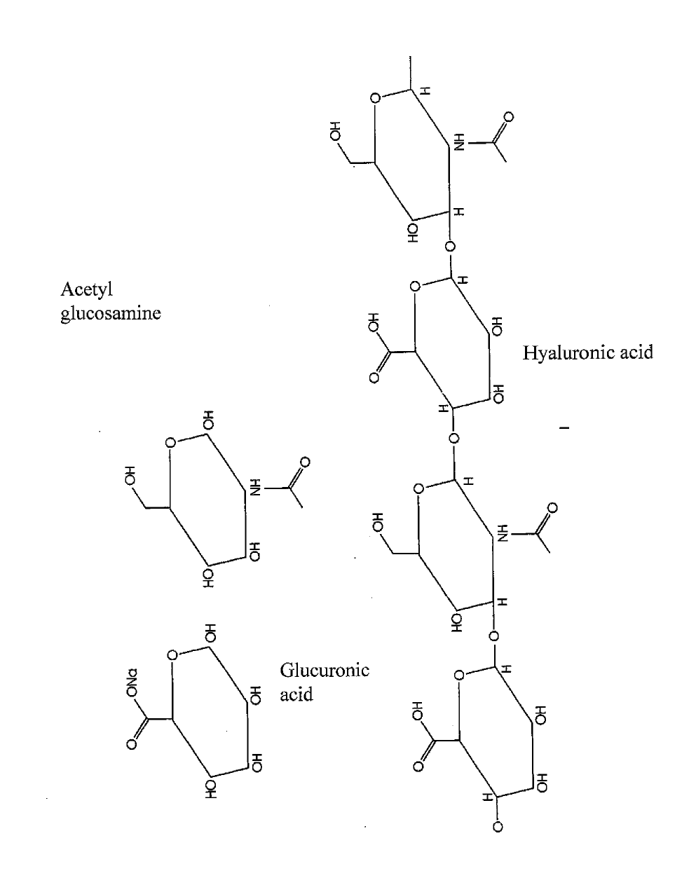

### CROSS-REFERENCE TO RELATED APPLICATIONS

This is a divisional of patent application Ser. No. 12/214,154 filed Dec. 4, 2008.

### STATEMENT REGARDING FEDERALLY SPONSORED RESEARCH OR DEVELOPMENT

Not Applicable

### REFERENCE TO A “SEQUENCE LISTING”, A TABLE, OR A COMPUTER PROGRAM LISTING APPENDIX SUBMITTED ON COMPACT DISC

Not Applicable

### BACKGROUND OF THE INVENTION

1. Field of the Invention

The invention concerns a combination of active ingredients for cosmetic preparations for external application to the skin, especially skin creams.

2. Description of Prior Art including Information Disclosed under 37 CFR 1.97 and 1.98

It is known from WO 2006/020994 A2 how to employ hyaluronic acid for injection into the skin as an active substance. Hyaluronic acid can bind a multiple of its own molecular weight in water and therefore constitutes an important moisture reservoir in the skin. Hyaluronic acid is constantly formed in the skin cells, and the natural synthesis of hyaluronic acid diminishes with age, which is one of the major causes of wrinkle formation in skin. The formation of wrinkles is counteracted by the injection of hyaluronic acid.

The problem on which the invention is based is to create a combination of active ingredients of the kind mentioned at the outset, which is distinguished by an improved deep action in the skin.

This problem is solved according to the invention with the features of the invention.

### BRIEF SUMMARY OF THE INVENTION

In accordance with the present invention, a method of improving the skin penetration of a cosmetic preparation for external application to the skin is provided which comprises:
(a) combining first and second active substances chosen from the group consisting of hyaluronic acid and derivatives thereof, the first active substance being at least 10 weight percent of the combination of the active substances in long-chain form with a molecular weight of at least 1,000,000 daltons and the second active substance being at least 2 weight percent of the combination of the active substances in short-chain form with a molecular weight of at most 2,000 daltons, and
(b) applying the combination to the skin for cosmetic purposes.

The method includes the step of at least partially enveloping at least some of the active ingredients with one or more phospholipids.

The long-chain form is contained in at least 20 wt. % of the total active substances. Preferably, the long-chain form is contained in at least 5 wt. % of the total active substances.

The molecular weight of the portion of the active ingredients which is enveloped is at most 150,000 daltons.

At least one of the active substances includes at least one alkaline metal. Preferably, the alkaline metal is sodium.

At least one of the active substances contains at least one alkyl thiosulfate group.

At least one of the active substances contains at least one silane group.

### DETAILED DESCRIPTION OF THE INVENTION

The combination of active ingredients of the invention has hyaluronic acid and/or at least one hyaluronic acid derivative. This should supplement the natural hyaluronic acid depots in the skin, to prevent signs of aging such as wrinkle formation. Hyaluronic acid is a polymer which can be used in different molecular weights. The higher the molecular weight of the hyaluronic acid, the better the hydrating action. This increased hydrating action, however, comes at the cost of the deep action of the hyaluronic acid, since bigger molecules necessarily have only a slight depth of penetration into the skin. Long-chain hyaluronic acid therefore remains essentially on the skin surface, so that it cannot exert any deep action. While short-chain hyaluronic acid molecules have less hydrating ability, they can nevertheless penetrate deeper into the skin than the long-chain forms. According to the invention, long-chain hyaluronic acid or its derivatives with a molecular weight of at least one million daltons are used in combination with short-chain hyaluronic acid or its derivatives with a molecular weight of not more than two hundred thousand daltons. The mass unit of the dalton corresponds to one twelfth the mass of the carbon isotope ¹²C and roughly corresponds to the atomic unit of mass. Thanks to this combination, surprisingly, it has been possible to demonstrate a higher water delivery to deep layers of skin than is possible with short-chain hyaluronic acids alone. It is believed that the long-chain hyaluronic acid molecules on the skin surface have a positive influence on the physical properties of the short-chain hyaluronic acid molecules in deeper skin layers. However, this mechanism is not yet definitively explained. The indicated minimum values for the portions of long-chain and short-chain forms of hyaluronic acid or its derivatives always refer to the combination of active ingredients and not to the entire cosmetic preparation. This means that the total of all hyaluronic acids or derivatives in an actual combination of active ingredients always amounts to 100 percent by weight. Other substances which are generally contained in cosmetic preparations, such as glycerin etc., should not be taken into account when determining the percents by weight of the combination of active ingredients itself. Finally, the combination of active ingredients contains aescin, a complex of the phytoactive substance of the horse chestnut. This active substance suppresses the depolymerization of hyaluronic acid, so that the chemical equilibrium is shifted to longer-chain hyaluronic acids. Furthermore, hyaluronic acid molecules survive longer and remain active in the deeper skin layers. In order to introduce the hyaluronic acid or hyaluronic acid derivative as effectively as possible in deeper skin layers, it is at least partly surrounded by phospholipids. The surface of the hyaluronic acid molecule or its derivative is covered by the phospholipids, so that the outwardly active surface corresponds to that of a lipid. In this way, the hyaluronic acid or its derivative can diffuse especially effectively into the skin with the help of hydrophobic forces.

Alternatively, it is advantageous for the combination of active ingredients to have acetyl glucosamine or one of its derivatives and glucuronic acid or one of its derivatives. Advantageously, the two substances are introduced into the combination in roughly molar parity. Due to biochemical processes within the skin, hyaluronic acids or derivatives with various chain lengths are formed in this way inside the skin, having the above described properties. Furthermore, it was found that an even deeper penetration of the combination of active ingredients into the skin is possible in this way.

An especially intense hydrating action results when the combinations of ingredients per claims 1 and 2 are used in combination.

Thanks to an at least 20% content of long-chain hyaluronic acid or derivative, a high hydrating ability is ensured, so that the combination of ingredients acts very effectively on the whole.

In order to reach especially deep lying skin layers, it is advantageous to have at least 5 percent by weight of the short-chain form of hyaluronic acid or derivative. This is especially important for the treatment of deep wrinkles.

In order to prevent the hyaluronic acid or its derivative being blocked from the skin on account of the surrounding phospholipid and the resulting larger diameter, it is advisable for the hyaluronic acid or its derivative to have a molecular weight of not more than 150,000 daltons.

Besides pure hyaluronic acid, alkaline metal-substituted derivatives of hyaluronic acid, acetyl glucosamine and glucuronic acid have proven to work well. Preferably, one hydrogen of the carbonate [sic!] group (COOH) is substituted by an alkaline metal. Reactive sodium has proven especially suited as the substituent.

It is advisable for the mentioned derivative to have at least one alkyl thiosulfate group. This functional group very easily forms disulfide bridges, which help with the stereoisomeric stabilization of the hyaluronic acid derivative.

Finally, it is advisable for the mentioned derivative to have at least one silane group.

Further benefits and features of the present invention shall be presented in the following detailed description by means of the accompanying figures, containing several sample embodiments of the present invention. However, it should be understood that the drawing only serves the purpose of representing the invention and does not limit the range of protection of the invention.

### EXAMPLES

A combination of active ingredients consists of:
43 wt. % of long-chain, sodium-substituted hyaluronic acid with a molecular weight of around 1.5 million daltons;
10 wt. % of short-chain, sodium-substituted hyaluronic acid with a molecular weight of around 100,000 daltons;
42 wt. % of a short-chain silanol derivative of hyaluronic acid with a molecular weight of around 100,000 daltons, which is surrounded by phospholipids, and
5 wt. % of aescin, a complex from the phytoactive substance of the horse chestnut.

The above-mentioned combination is reacted with water, containing 5.5% of active substance combination, 5% glycerin and 89.5% water. This yields a gel with a viscosity that enables a pumping in conventional gel dispensers. When this gel is applied in wrinkles of the skin, one gets an immediate smoothing effect. Furthermore, after only a few minutes there is already a demonstrable moistening of the deeper skin layers, going beyond the sum of effectiveness of the individual active substances.

In another formulation, 5.5% of the combination of active substances is mixed with 5% glycerin, 60% water, 30% oils or waxes, and 4.5% emulsifiers and other additives. One gets a cream which is easily distributed over the skin and soaking into the skin, by which moisture can be supplied to the skin over a large area.

As a couple of sample embodiments of the present invention are not [sic!] shown and described, it is to be understood that many changes and modifications of these described sample embodiments are possible without leaving the essential notions and range of protection of the invention, which is set forth by the claims.

While only a limited number of preferred embodiments of the present invention have been disclosed for purposes of illustration, it is obvious that many modifications and variations could be made thereto. It is intended to cover all of those modifications and variations which fall within the scope of the present invention, as defined by the following claims.

What is claimed is:

1. The method of improving the skin penetration of a cosmetic preparation for external application to the skin which comprises:
(a) combining first and second active substances chosen from the group consisting of hyaluronic acid and derivatives thereof, said first active substance being at least 10 weight percent of the combination of said active substances in long-chain form with a molecular weight of at least 1,000,000 daltons and said second active substance being at least 2 weight percent of the combination of said active substances in short-chain form with a molecular weight of at most 2,000 daltons, and
(b) applying said combination to the skin for cosmetic purposes.

2. The method of claim 1, including the step of at least partially enveloping at least some of said active ingredients with one or more phospholipids.

3. The method of claim 1, wherein the long-chain form is contained in at least 20 wt. % of the total active substances.

4. The method of claim 1, wherein the long-chain form is contained in at least 5 wt. % of the total active substances.

5. The method of claim 2, wherein the molecular weight of the portion of said active ingredients which is enveloped is at most 150,000 daltons.

6. The method of claim 1, wherein at least one of the active substances includes at least one alkaline metal.

7. The method of claim 6, wherein the alkaline metal is sodium.

8. The method of 1, wherein at least one of the active substances contains at least one alkyl thiosulfate group.

9. The method of claim 1, wherein at least one of the active substances contains at least one silane group.
